(19) United States
(12) Patent Application Publication
(10) Pub. No.: US 2023/0065280 A1
(43) Pub. Date: Mar. 2, 2023

(54) MEDICAL PRODUCT FOR DELIVERY OF A DRUG WITH AN INTENSIFIED EFFECT

(71) Applicants: INNORA GESELLSCHAFT MBH, Berlin (DE); CHARITÉ - UNIVERSITÄTSMEDIZIN BERLIN, Berlin (DE)

(72) Inventors: Ole GEMEINHARDT, Berlin (DE); Sebastian SCHURMANN-KAUFELD, Berlin (DE)

(73) Assignees: INNORA GESELLSCHAFT MBH, Berlin (DE); CHARITÉ - UNIVERSITÄTSMEDIZIN BERLIN, Berlin (DE)

(21) Appl. No.: 17/793,778

(22) PCT Filed: Jan. 20, 2021
(86) PCT No.: PCT/EP2021/051156

§ 371 Date: Jul. 19, 2022

(30) Foreign Application Priority Data
Jan. 20, 2020 (DE) 10 2020 101 197.0

(51) Int. Cl. A61F 2/958; A61L 31/16

(52) U.S. Cl. CPC A61F 2/958; A61L 31/16; A61L 2300/416; A61L 2300/606

(57) ABSTRACT

The invention concerns a medical device for at least periodical contact with diseased vessels such as a stent or a balloon catheter, for example, as well as a method for coating it with a defined solution.

In accordance with the invention, a restenosis inhibitor is disposed on an outer surface in an active substance concentration of more than 4 μg/mm².

The invention relates to a selection of products for more effective inhibition of a restenosis of arteries or other passages in the body following mechanical opening or widening of the respective lumens.

In the past 20 years, the problem of restenosis of arteries injured, for example, by angioplasty, atherectomy or stent implantation, using locally applied drugs has been very substantially alleviated. Prominent examples are drug-eluting stents (DES) for use in coronary arteries and the balloons of angioplasty catheters coated with paclitaxel (drug-coated balloons, DCBs).

DESs contain a very small dose of drug which is slowly delivered over a long period and which inhibits the cell proliferation caused following vessel wall injury, for example due to the forcible dilation of arteriosclerotically constricted arteries. exaggerated cell proliferation leads to thickening of the arterial wall and to narrowing of the arterial lumen, and therefore to a reduction in blood flow. Currently available DESs suffer from the disadvantage that they are permanent implants which permanently change the arterial segment which has been treated in respect of its structure and flexibility and makes subsequent interventions more difficult. A permanent implant is associated with an annual frequency of 0.4% to 2.0% of clinical events for the remaining lifespan following implantation, according to current knowledge. Preferred drugs for stents belong to the class of immunosuppressive macrolides, which are also known as limus substances, with the known agent being rapamycin (sirolimus). Stents coated with limus substances are currently used in most cases of narrowing of coronary arteries. As an alternative, and in difficult cases, bypass operations and, particularly in the case of narrowing of vessels already provided with a stent, DCBs are also available. If stents are to be avoided, in selected cases, DCBs may also be used instead of stents for the initial treatment of narrowing or occlusion of coronary arteries. In peripheral arteries, balloon catheters are preferably used; stents are only used when a satisfactory outcome cannot be obtained with the balloon.

DCBs also contain an active substance which inhibits the proliferation of cells, most preferably paclitaxel, which is known from tumour therapy. The advantage with DCBs is that they deliver the drug to the vessel wall, but the catheters per se remain in the vessel for only seconds to a few minutes, and at the end of the short-duration treatment are then removed in their entirety. Only a portion of the drug originally on the angioplasty balloon remains in the vessel wall. The vessel retains its original structure and flexibility, its function can recover and future interventions are not made more difficult. The disadvantage of DCBs over stent implantation is above all the lack of stabilisation of the cross section of the vessel. After dilating the vessel lumen with a balloon and release of the pressure in the balloon and removal thereof, elastic restenosis of the vessel lumen is not a rare occurrence, while a stent largely prevents the vessel wall from elastic retraction.

While coating balloon catheters and coating stents with proliferation-inhibiting drugs clearly reduces the frequency of restenosis of the lumens of the treated vessel segments, clinical results obtained up to now, particularly in peripheral arteries, have not been entirely satisfactory. Some of the treatments have not had the desired outcome, i.e. in less than 6-12 months, the vessel lumens constrict again despite treatment with the products coated with drugs to such an extent that another treatment is required. The frequency of this failure of the therapy is 5-10% on average. This means that the treated vessel segments often do not remain permanently open, and 2, 3 or more years following an initial successful treatment, renewed constriction or occlusion leads to further symptoms, which at best make it necessary to repeat the intervention; in more unfortunate cases, they can no longer be removed.

Currently, macrocyclic immunosuppressants (also known as limus substances, for example sirolimus such as rapamycin, everolimus etc.) are primarily used on stents for coronary arteries; paclitaxel is used on balloons. It would be obvious to research more effective active substances. To this end, experiments have been carried out in the past with stents (Muni N I et al. 2005; Liistro and Bolognese, 2003). Similarly, cytostatic substances which are sometimes much more effective than paclitaxel were tested on balloons of balloon catheters, without success (Speck U et al., J Cardiovasc Surg 2016;57:3-11).

A plurality of balloon catheters coated with proliferation-inhibiting drugs have undergone clinical tests, and over all of the treated patients, on average, narrowing of the vessel lumen was reduced, but none of the coatings worked for all patients and all of the treated vessel sections (Anantha-Narayanan M et al. Catheter Cardiovasc Interv. 2019 Jul. 1; 94 (1):139-148). This assertion was in respect of a point in time 6-12 months after the treatment. This desired effect for the coating reduced after this point in time.

None of the DCBs which have been described up to now have met the requirements for an effect in as many patients as possible and over many years. More effective drugs for coating balloon catheters have not yet been discovered.

In this respect, an improvement in the effect of current approaches for minimally invasive local therapy of vessel constriction or occlusion is important and the aim of the present invention. The improvement is aimed at a reduction in the proportion of patients who, after mechanical widening or restoration of the vessel lumen, prematurely require a new treatment or in whom the initial success of the treatment is lost after a few years.

The objective of the invention is therefore to obtain a more reliable, intensified and/or longer-lasting openness of the constricted vessel in a single treatment, in particular with the use of the preferred active substance paclitaxel. The concentration of the active substance (paclitaxel) or the quantity of active substance (paclitaxel) in the vessel wall immediately after the treatment is observed and measured as a prerequisite to achieving this aim and as a factor which can readily be measured experimentally.

This objective is achieved by means of a medical device and coating thereof with a coating solution with the features of the independent claims. In this regard, in a first aspect, the invention concerns a medical device for at least periodical contact with diseased vessels, comprising an elongated hollow body with an outer surface, wherein an active substance or active substance mixture comprising a restenosis inhibitor which is specific for delivery onto the vessel wall is disposed on the outer surface. In accordance with the invention, the active substance or the active substance mixture is present, at least in regions, in a loading (or surface density) of more than 4 μg/mm², in particular more than 5 μg/mm², in particular ≥6 μg/mm² on the surface.

In the text below, the term “outer surface” should be understood to mean a surface of the hollow body which faces away from the internal diameter or inner lumen of the hollow body, or which faces an inner wall of the vessel during its intended use. Because of the different sizes of the regions carrying the drug or the coating, the loading on the medical product is given in μg/mm² (=“loading density”); the total quantity of the drug on a medical product is given as the “dose/medical product”. The dose in experimental or clinical use can be raised by using more than one of these products.

The subject matter of the invention pertains to a selection invention. Balloon catheters coated with drugs for the inhibition of vessel constrictions following lumen dilation using mechanical methods was investigated for the first time approximately 20 years ago and has been clinically proven since approximately 2003. While the desired inhibition of a restenosis of the treated arterial sections of the arteries of the heart and some peripheral vessels is beyond doubt, it has to be accepted that an effect is not observed in all patients and all of the treated arteries, or it does not last for a long period. Despite testing various active substances, compositions for the coatings and the coating methods, nothing has changed in this respect.

Obvious ways of improving the efficacy or duration of efficacy such as raising the dose, were not followed up, partly in respect of concerns about compatibility, partly because of difficulties with adhesion and the layer thickness on the surfaces of suitable medical products.

The possible variables have been known for a long time, but a suitable selection which is necessary for achieving the objective has not yet been made available.

Thus, current actually known products consistently have a lower dose than is now claimed in the present invention, for the aforementioned reasons, namely compatibility concerns and coating capability (sufficient adhesion, layer thickness and therefore suitability for use). In addition, there are no examples of doses above 3.5 μg of active substance/mm² in the patent literature or of clinical use in the literature. In contrast, in the light of animal experiments, manufacturers have introduced lower doses than the originally selected and tested dose of 3 μg/mm².

Many publications warn against paclitaxel and in fact teach the opposite to the teaching of the invention as regards the increased coating and higher dose with the preferred active substance paclitaxel which has been found to be possible and useful.

Surprisingly, it has been shown that the action of DCBs comes dose to the target of a reliable and durable effect by means of a suitable selection of the balloon material and the coating. In this regard, it is easy to measure that a higher active substance density of more than 4 μg/mm², in particular more than 5 μg/mm² compared with the standard, for the same contact time with a vessel wall, leads to a significantly higher rate of transfer and therefore to a higher concentration of active substance in the vessel wall.

In order to achieve this aim, the use of a higher dose has to result in a larger quantity of drug at the target site, and the increase in the amount of active substance which is introduced into the target tissue leads to a greater effect. Among other things, an increase in the dose requires that the drug is tolerated well.

The usual doses on balloons with respect to the balloon surface area, because long balloons with a large diameter for the treatment of long segments of larger vessels naturally require more active substance than small segments of vessels with small lumens. The originally introduced dose of 3 μg of paclitaxel/mm² of balloon surface was at the time the maximum possible loading of the smooth balloon membrane which could be made. The products which have been available up to now contain rather smaller doses, in many cases only 2 μg/mm² of balloon surface for the active substance paclitaxel; the highest dosed product is 3.5 μg of paclitaxel/mm². Publications indicate concerns regarding the compatibility of the active substance paclitaxel on balloons compared with the less frequently used sirolimus (Wessely et al. 2006) or point out the risk of embolisms due to paclitaxel which is washed away distally and has low solubility in water (Gongora et al., 2017). Recently, a study was published which suggests an increased mortality rate following the use of paclitaxel-coated medical products, and also a warning to keep the dose of paclitaxel low.

A high dose means a dose which is sufficient to reduce the fraction of vessels which narrow again rapidly with respect to the total number of treated vessels and to extend the period for which the treated vessel segments remain open. In the case of paclitaxel and sirolimus, this dose is ≥5 μg/mm² of the surface of the medical product, preferably ≥6 μg/mm² and particularly preferably ≥10 μg/mm².

All advice is that a substantial increase in loading conventional balloon catheters with paclitaxel would compromise compatibility, and that a substantial increase in the loading of conventional balloon catheters with any active substance is difficult because of the many technical, drug and physiological problems if these catheters are to be used in patients.

Increasing the dose on a balloon surface pre-supposes that the increased quantity of drug adheres sufficiently well and is not lost during the folding of the balloon which is necessary.

Furthermore, the higher dose on the balloon must result in a higher quantity/concentration of the active substance in the arterial wall, which is not obvious because an increased quantity of active substance adheres less well to the balloon surface and could be lost on its way to the treatment site, and the take up capacity of the vessel wall during the short period of balloon inflation might be limited for the drug.

Nevertheless, the aforementioned aims are achieved with the novel combination of components and methods for balloon coating which are known per se.

Active substances or drugs which may be considered are highly lipophilic, substantially water-insoluble and highly effective drugs which will bind to any tissue component. Drugs are described as lipophilic when they have a butanol: aqueous buffer at pH 7 distribution coefficient=0.5, preferably=1, and particularly preferably=5, or an octanol: aqueous buffer pH 7 distribution coefficient=1, preferably=10, particularly preferably >50. As an alternative or in addition, the drugs should bind reversibly and/or irreversibly to cell components to an extent of >10%, preferably >50%, particularly preferably >80%. Substances for inhibiting cell proliferation or in addition inflammatory processes or antioxidants are preferred, such as paclitaxel and other taxanes, rapamycin and related substances, tacrolimus and related substances, corticoids, sex hormones (oestrogens, oestradiol, antiandrogens) and related substances, statins, epothilones, probucol, prostacyclins, angiogenesis inducers etc. The substances are preferably in the form of a dry solid substance or as an oil on the surfaces of the various medical products. Particles with as small a size as possible are preferred (majority <5 μm, preferably <1 μm, particularly preferably <0.1 μm); crystalline structures are particularly preferred.

The dose is aimed at the desired effect and the efficacy of the drug employed. It can be as high as 6 μg/mm², but this does not constitute an upper limit. For the coating in accordance with the invention, wires like those used to guide catheters are used; needles and catheters or parts of catheters which are pressed against diseased tissue for at least a short period using pressure are envisaged. The length and diameter of the regions of the catheter or balloon intended for the pharmacotherapy is not of great significance for the application, because the dose is calculated in μg of active substance/mm² of surface. As an example, for coronary dilation balloons, a range of <2-4 mm in diameter and 1.0-4.0 cm in length is usual. For other vessels, balloons of up to >20 mm diameter and lengths of up to >20 cm may be employed. The surfaces to be coated may be smooth (i.e. without a particular structure for receiving the active substances), roughened or provided with structures in any manner, wherein special surface structures are not a prerequisite for adhesion of the active substances, but do not inhibit adhesion. The adhesion of the active substances to the balloon surfaces is exclusively brought about by the choice of suitable solvents and, if appropriate, additives which influence adhesion. Surprisingly, it is even securely attached to extremely and completely smooth balloon surfaces.

All of the surfaces may additionally have been coated or be coated with substances which improve the glide properties of the products, which prevent clotting of blood on the surface or improve other properties of the medical products without the materials used for the coating being discharged into the environment and without the coating substantially restricting delivery of the active substances for the treatment of the target tissue, and therefore the efficacy.

Thus, in one embodiment of the invention, the medical device preferably further has auxiliary substances on the outer surface.

Good adhesion to the surfaces of the catheter, needles or wires while improving the take-up into the tissue is obtained by incorporating highly lipophilic, low water-solubility active substances into a readily water-soluble matrix substance. Suitable matrix substances are low molecular weight (molecular weight <5000 D, preferably <2000 D) hydrophilic substances such as contrast agents used in vivo and dyes for various diagnostic methods in medicine, sugar and related substances such as sugar alcohols, low molecular weight polyethylene glycols, biocompatible organic and inorganic salts such as benzoates, salts and other derivatives of salicylic acid, etc. Iodated radiographic contrast agents and paramagnetic chelates are examples of contrast agents; examples of dyes are indocyanine green, fluorescein and methylene blue. Auxiliary substances may also function to improve the storage properties of the products, or function to bring about supplementary pharmacological effects or be used for quality control.

Thus, the surface of the device in accordance with the invention advantageously comprises or consists of auxiliary substances which contain organically bound iodine, preferably iopamidol, iomeprol, iopromide and/or iohexol, as well as urea, magnesium salts, in particular magnesium stearate, dexpanthenol, lipophilic antioxidants, in particular nordihydroguaiaretic acid, resveratrol and/or propyl gallate or combinations thereof.

In a particularly preferred embodiment of the invention, an organically bound iodine is disposed on the outer surface, at least in regions, in a loading density in the range from 0.1 μg/mm² to 0.8 μg/mm², preferably 0.1 μg/mm² to 0.5 μg/mm². This corresponds to a loading density of iodine-containing organic substance of approximately 0.2 μg/mm² to 1.6 μg/mm² or approximately 2% to 20% iodine with respect to the active substance in the range 4-6 μg of active substance/mm² of surface area.

It has been shown that the reduced loading density of the auxiliary substance compared to the standard quantity results in an improved transfer of the active substance onto the vessel wall.

In a further embodiment, the pharmaceutical active substances may be adsorbed onto particles or be applied with a low molecular weight matrix onto the surfaces of suitable medical products.

Again, suitable particles are known biocompatible diagnostic compounds such as ferrite and various contrast agents for sonography.

Balloon catheters are formed from very thin plastic tubes by expanding a segment from 1 to >20 cm in length. The expanded, very thin-walled balloon membrane is then placed into a plurality of folds which are disposed longitudinally to the catheter axis and wound securely around the catheter axis so that in the folded state, the subsequently expanded region has only a minimally larger diameter than the usual catheter. The tight folding of the balloon sheath is the prerequisite for passing the balloon catheter through loading connectors, guide catheters and, for example, greatly narrowed sections of blood vessels without problems.

In a preferred embodiment of the invention, the outer surface is, at least in sections, a non-elastic compression-resistant membrane, wherein the membrane is preferably the balloon of a balloon catheter as described above.

Preferred catheter materials are polyamides, polyamide blends and copolymers, polyethylene terephthalate, polyethylene and copolymers.

In a particularly preferred embodiment, the membrane, i.e. the catheter material, comprises or consists of polyamide, polyether block amides (PEBAX, vestamid), polyethylene, polyethylene terephthalate or their copolymers and/or blends.

In a preferred embodiment, the balloon can be inflated to more than 15 bar, in particular more than 30 bar. This can in particular be achieved by means of the aforementioned materials.

In a further aspect, the invention concerns the use of a solution for coating a medical device for the treatment of diseased vessels, in particular for the production of the device in accordance with the invention. The solution used in this regard comprises a solvent, a restenosis-inhibiting active substance and an auxiliary substance comprising organically bound iodine forming a matrix for the active substance. In accordance with the invention, the auxiliary substance contains the organically bound iodine in an amount in the range from 1.2% to 12.5% by weight, preferably 2.5% by weight to 12.5% by weight with respect to the active substance in the solution. At a loading density for the active substance of 4, this advantageously results in a loading density for the iodine in the range from 0.1 μg/mm² to 0.75 μg/mm², preferably from 0.1 μg/mm² to 0.5 on the outer surface.

It has been shown that a medical device coated with the solution in accordance with the invention, in particular a balloon catheter, delivers more active substance over the same period onto the vessel wall than the known coated devices of the prior art.

Advantageously, the organically bound iodine is present in the solution as iopromide, iopamidol and/or iomeprol.

In a preferred embodiment of the invention, the solution contains the active substance, in particular paclitaxel or sirolimus, in a concentration in the range from 100 mg to 200 mg in 5 mL of solution.

Examples of suitable solvents are methanol, ethanol, isopropanol, ethyl acetate, diethyl ether, acetone, tetrahydrofuran, dimethyl sulphoxide, dimethyl formamide, water or mixtures thereof. The selection of the solvent is made as a function of the solubility of the active substances and additives as well as wetting of the surfaces to be coated and the effect on the structure of the coating which is left behind following evaporation of the solvent and particles, its adhesion to the surface and the transfer of active substance into the tissue over very short contact times.

Particularly advantageously, the solution contains acetone, water and/or ethanol as the solvent, wherein the solvent mixture contains 3% to 25% by volume, in particular 5% to 15% by volume of water.

The starting point for the experiments are preferably formulations in accordance with WO 2004/028582 A1, Example 7, preferably solution B. As opposed to the cited example, the proportion of Ultravist 370 was reduced from 100 μL/5 mL of coating solution to 5-50 μL/5 mL of solution mixture; the reduced volume of Ultravist resulted in 3.8-38.45 mg of iopromide/150 mg of paclitaxel or 1.85-18.5 mg of organically bound iodine/150 mg of paclitaxel, particularly preferably 5-20 μL of Ultravist 370/5 mL of solution mixture. 100 μL of Ultravist 370 contains 76.9 mg of the radiographic contrast agent iopromide, corresponding to 37 mg of organically bound iodine.

In the solvent for paclitaxel and Ultravist, the fraction of water compared with Example 7 of WO 2004/028582 A1 with solution B was raised from <1.3% by volume to 3-25% by volume, particularly preferably 5-15% by volume. Instead of the radiographic contrast agent Ultravist 370, an aqueous solution of the contrast agent iopromide, which is contained in Ultravist, could be used. Similarly, comparable other contrast agents may be used, such as Isovue™ 370 with iopamidol as the contrast agent, or Iomeron 400 with iomeprol as the contrast agent, or other comparable products in available concentrations.

Application may, for example, be by means of dipping, coating, application by means of volumetric measuring devices or by spraying at respectively different temperatures and, if appropriate, steam saturation of the solvent in the atmosphere. The procedure may be repeated multiple times, if appropriate also using different solvents and auxiliary substances.

In a particularly preferred embodiment, the balloons are coated in the inflated state, in particular with a canula with a defined volume of the active substance solution. After drying and folding, the balloons may be treated with dry lubricant, for example magnesium stearate powder, or may be dipped for a very short period into an aqueous suspension of magnesium stearate or a suspension or solution of another biocompatible lubricant which can degrade in the human metabolism or can be excreted by human beings, or can be sprayed with a suspension or solution of this type or be wetted in some other way. After this step, the balloons are dried again, provided with a protective sleeve and sterilized using EO. The end product is a sterile, properly packaged high-dose paclitaxel-coated balloon catheter which is suitable and authorised for use with human beings.

Supports for the drug or drugs are conventional balloon catheters with proximal handles, a catheter shaft with a wire lumen and a liquid lumen, a proximal handle with a connector for a syringe and insertion port for a guidewire and a distal balloon with a smooth or structured surface produced from a thin, non-elastic or barely elastic compression-resistant membrane produced from polyamide, for example, a polyether block amide (for example PEBAX or vestamid), polyamide blends and copolymers, polyethylenes, polyethylene terephthalate, for example, in sizes which are suitable for the treatment of arteries of all types, for example in the heart, in the skull, in the extremities or elsewhere in the body. “Compression-resistant” means that the balloons can be inflated to 4->30 bar without bursting. The balloons may contain elements produced from other materials, for example metals or plastics, which endow the balloon membrane with additional compressive strength, change the shape of the inflated balloon or exert an effect on the tissue it lies against upon inflation, for example it scratches, cuts or influences the tissue with heat or electrical pulses.

For the coating, the active substances and, if appropriate, the auxiliary substances and additives as well, are dissolved or suspended in organic solvents with or without the addition of water. Preferred solvent mixtures contain acetone, ethanol and water; preferred additives are radiographic contrast agents such as iopamidol, iopromide or iohexol, but also other conventional auxiliary substances for coating balloon catheters such as urea, magnesium salts which, in proportions of ≤20% by weight of the active substance, have a positive influence on their adhesion to the surfaces of the medical products and their release at the target site and/or promote the transfer of the active substances into the tissue. Additives of active substances or auxiliary substances which alleviate inflammatory reactions of the tissue and/or which accelerate healing are particularly preferred; examples are dexpanthenol and corticoids.

The efficacy of drugs on medical products can be intensified without raising the dose by improving transfer into the tissue to be treated. This is particularly important for medical products which remain at the target site for only a short period. Examples of this are balloon catheters which are inflated in a vessel, although inflation completely interrupts the flow of blood and therefore, for example in coronary arteries or in fact in arteries supplying the central nervous system, they are deflated and removed after a very short period. The transfer of the drug from a balloon of a balloon catheter into the vessel wall of coronary arteries was, according to the first publication from Scheller et al., 2004, on average 8.7±4.9% of the total dose on the balloon as long as no stent had been implanted or was implanted. In a more recent study (Speck et al., 2018), the transfer was given as 7.8±3.4% for coronary arteries and 7.1±6.1% of the dose for leg arteries in pigs.

An increase in the proportion of the active substance delivered to the tissue to be treated is an aim in the development of novel DCBs. To this end, the composition of the coating and the mode of coating has been varied in multiple ways by various firms, but clearly without substantial improvement to the efficacy (Anantha-Narayanan M et al. Catheter Cardiovasc Interv. 2019 Jul. 1;94 (1):139-148; Meredith IT, TCT2019). On the clinical side, handling of the balloon catheter has been improved in that the vessel segments to be treated are carefully prepared beforehand and the period for inflation of the balloons has been lengthened where possible.

Entirely surprisingly, it has been found that drug-tissue transfer is improved by selecting a suitable balloon membrane in combination with selected coating formulations and coating modes, resulting in a reproducibly high transfer of drugs into the arterial wall. This is the case, for example, for polyether block amide membranes, in particular those marketed with the trade names PEBX or vestamid in combination with formulations with lipophilic antioxidants such as NDGA (nordihydroguaiaretic acid) and propyl gallate, for example, while other formulations (without auxiliary substances or with other auxiliary substances) exhibited no differences between PEBAX and, for example nylon membranes in the case of the transfer of active substances into the arterial wall (Example 3, Table 3).

Membranes of balloon catheters have different stabilities when raising the pressure in the balloon. Elastic membranes produced from elastic materials such as latex or polyurethane exist which are inflated at low pressures and adapt to the shape or the diameter of vessels or body cavities. A further group is formed by the balloons conventionally used for angioplasty with largely dimensionally stable membranes, for example produced from nylon or PEBAX (non-compliant or semi-compliant) which are used to dilate constricted arteries and can tolerate pressures of up to approximately 15, maximum 20 bar. Finally, for very solid vessel narrowing, for example in highly calcified vessels or in arterio-venous shunts for dialysis in patients with insufficient kidney function, balloon catheters are used which have balloons which can tolerate substantially higher pressures. In the case of stenoses the dilation of which require high pressures, these can be dilated by means of a high pressure balloon (suitable for pressures of up to approximately 20-40 bar) in the context of “vessel preparation” and then counteract excessive scar formation stimulated by the injury to the vessel by subsequent treatment with a DCB. By using the DCB, restenosis of the lumen is reduced. Furthermore, the durability of the treatment is not good in many cases (Steiner K, Endovascular Today June 2016; Karnabatidis TCT 2013).

Earlier studies indicated that the inflation pressure during transfer of the drug into the vessel wall (Bienek et al. Catheter Cardiovasc Interv. 2020 February;95:319-328)) and its effect (Cremers et al., Clin Res Cardiol. 2012; 101: 385-91) had no significant influence. At low inflation pressures (2 bar) for a DCB, an inhibition of lumen narrowing was found in animal experiments which was similar to that with a higher inflation pressure (12 bar). With another balloon, even at very low pressures (<2 bar), 13.9±6.4% of the dose was transferred from the balloon into the vessel wall.

Experiments with coated high pressure balloons produced the opposite. A significant increase in the transfer of drug into the treated arterial wall was observed over the conventional DCBs inflated to approximately 10 bar. Because no substantial difference was found between drug transfer at a very low inflation pressure and higher inflation pressure and the effect of the drug upon inflation of the balloon with approximately 2 bar and with approximately 12 bar, the observed increase in drug transfer into the vessel wall after using balloons with a very high inflation pressure was surprising. The increased drug transfer into the tissue would mean that even in vessel constrictions which are difficult to treat and which can only be widened with a large force, for example calcified arteries, a substantial inhibition of restenosis would be expected.

In a preferred application, balloon catheters function to dilate narrowed or occluded arteries. Balloon catheters were coated with drugs in order to prevent restenosis occurring shortly after dilation of the arterial lumen due to excessive cell proliferation. The properties and treatment outcomes for the first balloon catheters coated with drugs in this manner were published in 2004 (experimental, Scheller et al.) and 2007 (clinical, Scheller et al.). With the coating of that time, only 8.7±4.9% of the dose went from the balloon into the arterial tissue (Scheller et al., 2004). This introduction into the arterial wall, which is decisive for the effect, could be substantially raised even with respect to the current commercial product from B. Braun.

### EXAMPLE 1

Increasing paclitaxel (Ptx) transfer from coated, EO-sterilised balloon into the treated arterial tissue (column 6) by changing the coating: reduction of auxiliary substance (column 4), increasing water content of coating solution (column 3) and coating balloon in expanded state (column 2). All balloons with approximately 3 μg paclitaxel/mm² balloon surface. Methods: Experiments on coronary arteries from pigs; for method, see Scheller et al. 2004, Speck et al. 2018.

| | Column | | | | | | |
|---|---|---|---|---|---|---|---|
| | | | 3 | 4 | | | 7 |
| | | 2 | Water | Auxiliary | 5 | 6 | Ptx |
| | | State of | content of | substance | Paclitaxel | Ptx | remaining |
| | | balloon | coating | (iopromide) | on the | transferred | on used |
| Group | 1 | during | solution | % by weight | balloon | into the arterial | balloon |
| no. | Coating | coating | (v/v %)* | of paclitaxel | [μg] | tissue % of (5) | % of (5) |
| A | experimental | inflated | 1.3% | 0 | 625 ± 111 | 16.0 ± 11.8 | 23.1 ± 10.2 |
| B | experimental | inflated | 1.4% | 10 | 680 ± 326 | 19.1 ± 11.1 | 21.0 ± 14.7 |
| C | experimental | inflated | 10.0% | 50 | 745 ± 31 | 29.9 ± 13.5 | 15.0 ± 10.0 |
| D | experimental | inflated | 1.3% | 50 | 765 ± 66 | 25.9 ± 11.7 | 16.8 ± 4.7 |
| | paccocath | | | | | | |
| | coating | | | | | | |
| Circ. | paccocath | folded | | 50 | 550 | 8.7 ± 4.9 | 7.9 ± 2.6 |
| 2004 | | | | | | | |

By means of the balloon catheters coated in accordance with Example 7B of DE 10244847, on average 8.7% of the active substance paclitaxel was transferred into the arterial wall, the selected new coatings resulted in a significantly higher proportion of the dose in the vessel wall (19.1-29.9%), wherein the version without auxiliary substance (group A) remained unconsidered, because previous balloon coatings with paclitaxel without auxiliary substances had proved to be less effective in patients having regard to the desired inhibition of restenosis.

### EXAMPLE 2

### Increase of Dose on Balloon

Balloon catheters from Acotec, balloon dimensions 4×40 to 7×40 mm, were coated with paclitaxel using a composition with 10% by volume water and 10% by weight iopromide with respect to the active substance in the solution and inflated in the A. iliaca interna or A. femoralis of domestic pigs for 1 min (for method, see Scheller et al. 2004, Speck et al. 2018).

Column (1): paclitaxel (Ptx) in μg/mm² of balloon surface, with respect to the balloon surface area, because the balloons have or may have different lengths for the treatment of different arteries and sections of arteries and (for applications other than those described here) a different diameter. Columns 2-4: paclitaxel in the treated arterial wall (segment of the A. iliaca interna or A. femoralis of domestic pigs, approximately 3 months old, approximately 25 kg in weight) 10-40 min after balloon deflation; column 5: proportion of original paclitaxel dose found on the balloon after removal from the animal (see column 1).

Line 4, column 1: number of measured balloon catheters; columns 2-4: number of arteries investigated; column 5: number of balloons used.

Line 5, columns 2-5: in each artery, a balloon coated with paclitaxel with a 3 μg/mm² balloon surface was inflated for 1 min, then deflated and removed.

Line 6, columns 2-5: in each artery, two balloons coated with paclitaxel of the same type as in line 5 were inflated one after the other in the identical arterial segment in order to transfer more drug into the arterial wall.

Line 7: as in line 5, only one coated balloon was inflated in each arterial segment, but this had been coated with twice the quantity of paclitaxel.

| Line | Column no. | 1 | 2 | 3 | 4 | 5 |
|---|---|---|---|---|---|---|
| 1 | dose group | Paclitaxel on | Ptx transfer into | Ptx transfer into | Ptx transfer into | Ptx remaining |
| | | one balloon | arterial wall | arterial wall | arterial wall | on used balloon |
| 2 | | | total | concentration in | proportion of | proportion of |
| | | | | tissue | dose | dose |
| 3 | | [μg/mm²] | [μg] | [μg/g] | [% of column 1] | [% of column 1] |
| 4 | n | | 8 | 8 | 8 | 8 |
| 5 | standard | 3.06 ± 0.09 | 379 ± 265 | 787 ± 738 | 15.5 ± 10.3 | 9.5 ± 2.4 |
| | dose | | | | | |
| | 3 μg/mm² | | | | | |
| 6 | 2 balloons, | 3.06 ± 0.09 | 694 ± 492 | 1287 ± 619 | 14.6 ± 10.9 | 9.8 ± 3.1* |
| | each with | | | | | |
| | 3 μg/mm² | | | | | |
| 7 | 1 balloon, | 5.88 ± 0.20 | 796 ± 564 | 1957 ± 1472 | 16.9 ± 11.5 | 5.5 ± 1.2 |
| | 6 μg/mm² | | | | | |

Result and conclusion: With the dose of 3 μg/mm² of balloon surface, the expected values for this type of coating in respect of the drug transfer into the tissue and finally the small amount remaining on the balloon were measured. The use of 2 identical catheters with inflation in exactly the same segment of the vessel resulted in almost double the quantity of drug in the artery. Doubling the dose on the balloon had at least the same effect. At least double the quantity of drug was transferred into the arterial wall. This should be understood to be an indication of increased efficacy.

### EXAMPLE 3

### Influence of Balloon Membrane and Composition of the Coating on the Transfer of Paclitaxel and Sirolimus onto the Arterial Wall in Pigs

Balloons of balloon catheters were coated as stated above with paclitaxel-containing (Ptx) or sirolimus-containing formulations in the expanded state, folded and sterilised with ethylene oxide; the loss of active substance was measured during passage through a haemostatic valve, a guide catheter filled with blood (length 1 m) and with a 1 minute dwell time in blood (line 5), lines 6-8 show results of the experiments on coronary arteries in pigs; for method see Example (1). PTCA=percutaneous transluminal coronary angioplasty, Pebax=polyether block amide, NDGA=nordihydroguaiaretic acid, BHT=butylhydroxytoluene.

| | | | | | | | PTCA balloon catheter (Kossel | | |
|---|---|---|---|---|---|---|---|---|---|
| | | PTCA balloon catheter from MitrAssist Lifesciences Limited; | | | | | Medtech, Suzhou City, China) Nylon/ | | |
| No. | Catheter | Nylon/Pebax balloon membrane | | | | | Pebax balloon membrane | | |
| 1 | Internal experiment | IMTR 20180917 | | | IMTR20181021, | | IMTR20190304 | | |
| | no. | | | | | | | | |
| 2 | Balloon membrane | Nylon | PEBAX | n | PEBAX/ | n | Nylon | PEBAX | n |
| 3 | Formulation, active | D10d; Ptx, NDGA | D10d; Ptx, | | MA- PG2; Ptx, | | Sirolimus, BHT | | |
| | substance, auxiliary | | NDGA | | propyl gallate | | | | |
| | substance | | | | | | | | |
| 4 | Total active | 1.98 | 1.98 | 2 | 3.09 ± 0.09 | 4 | 4.8 ± 0.5 | 4.8 ± 0.3 | 3 |
| | substance on | | | | | | | | |
| | balloon, μg/mm² | | | | | | | | |
| 5 | Active substance | 10.3 ± 6.5 | 4.4 ± 5.4 | 4 | 16.8 ± 2.6 | 3 | 3.7 ± 4.0 | 0.7 ± 2.2 | 3 |
| | loss in haemostatic | | | | | | | | |
| | valve, % of dose | | | | | | | | |
| | (line 4) | | | | | | | | |
| 6 | Transfer into pig | 6.0 ± 3.4 | 26.2 ± 8.0 | 6 | 35.4 ± 8.0 | 9 | 19.0 ± 7.923 | 23.6 ± 12.9 | 6 |
| | arterial wall, | | | | | | | | |
| | coronary, % of | | | | | | | | |
| | dose (line 4) | | | | | | | | |
| 7 | μg/g of arterial | 116 ± 55 | 497 ± 191 | 6 | 834 ± 305 | 9 | 673 ± 339 | 815 ± 467 | 6 |
| | tissue | | | | | | | | |
| 8 | Remainder on | 30.1 ± 6.5 | 26.0 ± 2.5 | 6 | 4.2 ± 0.5 | 9 | 16.6 ± 4.8 | 16.5 ± 4.7 | 6 |
| | balloon, % of dose | | | | | | | | |
| | (line 4) | | | | | | | | |

Results and conclusions: The balloons consisted of nylon or Pebax, and were indistinguishably transparent and smooth. A substantially homogeneous coating of the balloon membranes was obtained in the range from approximately 2 to 5 μg/mm². On average over all experiments, approximately 10% of the dose was lost on passage through a haemostatic valve, a guide catheter filled with blood and the dwell time in the blood. The transfer of paclitaxel from formulations with the antioxidants NDGA and propyl gallate was unusually high at approximately 30% of the dose for the balloons with pebax membranes, both in a direct comparison with balloons produced from nylon membranes (6.0±3.4%) and also in comparison with data from the literature (Scheller et al., 2004: 7.9±2.6%, Speck et al. 2018: 7.8±3.4%). This led to high paclitaxel concentrations in the tissue (line 8). The substantially improved transfer of the active substance paclitaxel into the arterial tissue observed (in the combination of antioxidant and pebax balloon) is surprising and is an indication of intensified efficacy of this balloon catheter. The proportion of the dose which remained on the balloon (line 9) was not very different between pebax and nylon balloons, but much smaller when propyl gallate was used as the antioxidant.

### EXAMPLE 4

### Composition of a Coating in Accordance with WO 2004/028582 A1, Example 7, Optimized

81.1% by volume of acetone, 8.8% by volume of ethanol, 0.4% by volume of Ultravist 370, 9.7% by volume of water, 150 mg of paclitaxel on 5 mL of coating solution.

1. A medical device for at least periodical contact with diseased vessels, comprising an elongated hollow body with an outer surface, wherein an active substance or active substance mixture comprising a restenosis inhibitor which is specific for delivery onto the vessel wall is disposed on the outer surface, characterized in that the active substance or the active substance mixture comprises or consists of paclitaxel as the restenosis inhibitor and is present on the surface, at least in regions, in a proportion of ≥6 μg/mm², wherein the medical device is a balloon catheter, wherein furthermore, auxiliary substances are disposed on the outer surface, to be selected from a group consisting of organically bound iodine, urea, magnesium salts, dexpanthenol, iopamidol, iopromide, iomeprol, iohexol, magnesium stearate or combinations thereof.

2. (canceled)

3. (canceled)

4. The medical device as claimed in claim 1, wherein, in addition to the auxiliary substances, lipophilic antioxidants, in particular nordihydroguaiaretic acid, resveratrol and/or propyl gallate, are disposed on the outer.

5. The medical device as claimed in claim 1, characterized in that furthermore, an organically bound iodine is disposed on the outer surface at least in regions in a loading density in the range from 0.1 μg/mm² to 0.5 μg/mm².

6. The medical device as claimed in claim 1, characterized in that at least sections of the outer surface comprise a non-elastic, compression-resistant membrane, wherein the membrane is preferably constructed as a balloon of the device configured as a balloon catheter.

7. The medical device as claimed in claim 6, characterized in that, the membrane comprises or consists of polyamide, polyether block amide, polyethylene, polyethylene terephthalate or their copolymers and/or blends.

8. The medical device as claimed in claim 6, characterized in that the balloon can be inflated to more than 15 bar, in particular more than 30 bar.

9. A method for coating a medical device for the treatment of diseased vessels, comprising: coating the medical device with a solution comprising a solvent, a restenosis-inhibiting active substance and an auxiliary substance comprising organically bound iodine forming a matrix for the active substance,
wherein the auxiliary substance contains the organically bound iodine in a concentration in the range from 1.2% to 12.5% by weight, preferably 2.5% by weight to 12.5% by weight with respect to the active substance in the solution, and wherein the organically bound iodine is present as iopromide, iopamidol and/or iomeprol.

10. (canceled)

11. The method as claimed in claim 9, characterized in that the solution contains paclitaxel in a concentration in the range from 100 mg to 200 mg in 5 mL of solution.

12. The method as claimed in claim 9, characterized in that the solution contains acetone, water and/or ethanol as the solvent, and wherein the solvent contains 3% to 25% by volume, in particular 5% to 15% by volume of water.

13. A method for coating a medical device for the treatment of diseased vessels, comprising the following steps in the given order:
(a) preparing a solution comprising a solvent, a restenosis-inhibiting active substance and an auxiliary substance comprising organically bound iodine forming a matrix for the active substance, wherein the auxiliary substance contains the organically bound iodine in a concentration in the range from 1.2% to 12.5% by weight, preferably 2.5% by weight to 12.5% by weight with respect to the active substance in the solution, and wherein the organically bound iodine is present as iopromide, iopamidol and/or iomeprol;
(b) applying the solution to at least regions of an outer surface of the device, preferably as claimed in claim 1, by means of dipping, spraying or wetting with a volumetric measuring device; and
(c) drying the device.

14. The method as claimed in claim 13, characterized in that the device has an inflatable balloon and in step (b), an outer surface of the balloon is coated in the inflated state, at least in regions.

15. The medical device as claimed in claim 4, characterized in that furthermore, an organically bound iodine is disposed on the outer surface at least in regions in a loading density in the range from 0.1 μg/mm² to 0.5 μg/mm².

16. The medical device as claimed in claim 7, characterized in that the balloon can be inflated to more than 15 bar, in particular more than 30 bar.

17. The method as claimed in claim 11, characterized in that the solution contains acetone, water and/or ethanol as the solvent, and wherein the solvent contains 3% to 25% by volume, in particular 5% to 15% by volume of water.
